(19) United States
(12) Patent Application Publication
(10) Pub. No.: US 2006/0149209 A1
(43) Pub. Date: Jul. 6, 2006

(54) Absorbent article having improved bonding pattern

(75) Inventors: Gregory L Malchow, Oshkosh, WI (US); Paul J Datta, Appleton, WI (US); Tom R Belau, Neenah, WI (US); John F Condron, Neenah, WI (US); Melissa C Putzer, Oshkosh, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide Inc., Neenah, WI (US)

(21) Appl. No.: 10/524,717

(22) PCT Filed: Aug. 22, 2003
(86) PCT No.: PCT/US03/26248

§ 371 Date: Feb. 15, 2005

Related U.S. Application Data
(60) Provisional application No. 60/405,861, filed on Aug. 22, 2002.
(60) Provisional application No. 60/406,199, filed on Aug. 26, 2002.

(51) Int. Cl. A61F 13/15

(52) U.S. Cl. 604/389

(57) ABSTRACT

The present invention relates to improvements in bonding patterns for the attachment of components of disposable absorbent articles. For example, in order to provide attachment of an ear portion to a longitudinal side of the chassis of an article, there may be an attachment area The attachment area may include an overlapping area of chassis material(s) (outer cover and/or liner materials) and ear portion material. The over-lapping areas of chassis material and ear portion material are bonded together in such a way as to allow the bond pattern area to stretch from 8% to 30%. The bonding patterns of the invention may also be used to form the side seams and passive side bonds for articles that are donned as pants.

This patent application is a non-provisional patent application claiming priority to U.S. provisional patent application serial No. 60/405861 filed on Aug. 22, 2002.

### BACKGROUND OF THE INVENTION

The present invention is generally related to the assembly of components of disposable absorbent articles. The present invention also relates to the attachment of components such as ear portions to the main portion or chassis of a disposable absorbent article. Additionally, the present invention relates to bonding patterns that increase the durability and integrity with which components are attached to disposable absorbent articles.

Disposable absorbent articles have been known for a long time as personal care hygiene products. Disposable absorbent articles include infant diapers, is diaper pants, toddler training pants, swim pants, adult incontinence pads and briefs and women's sanitary pads. Such absorbent articles are designed and constructed to absorb and store solid and liquid bodily excretions such as urine, feces, menstrual fluid, or blood.

Nonwoven materials may be used extensively for the construction of disposable absorbent articles. Frequently, disposable absorbent articles are constructed of multiple nonwoven material components. For example, a disposable absorbent article may include a bodyside liner (also known as a topsheet) that contacts the wearer's skin in use, an absorbent core and an outer cover (also known as a backsheet) that is typically liquid impermeable. These three components run generally continuously with each other and together form what may be referred to as the “chassis” of the article. In addition, disposable absorbent articles may include components such as leg elastics, waist elastics, containment flaps, front waist ear portions, back waist ear portions and fastening systems for improving the fit and containment of the articles. These types of components are generally incorporated into the articles by attaching them to the chassis of the article. For example, the front waist ear portions and back waist ear portions may be attached to the chassis during manufacture.

The components of the disposable absorbent article used to secure the article to the intended wearer (e.g. ear portions and fasteners) tend to experience a significant concentration of stress during application of the article. If the concentration of stress exceeds the strength of the material, the components may become partially or completely detached from the article. The concentration of stress may be increased in regions or areas where multiple layers of materials are bonded together. An example of an area that may be subjected to increased stress forces during use of the article is the area where the back ear portions are attached to the back waist region of the chassis. Typically, ear portions are attached to the chassis of an article near the waist edge and along the longitudinal side edge of an article. With this example, the back ear portion will include at least one layer of nonwoven material that may be attached to at least the bodyside liner or the outer cover materials of the chassis. One way that the back ear portion may be attached to the chassis is by ultrasonic bonding. Premature failure at the attachment area, either where components are attached to the chassis or where components are attached to one another, may cause a negative impact on product appearance and/or product performance. For example, if the attachment of the fasteners fails, the article may not fit as intended. Poor fit may lead to a decrease in effectiveness of the absorbent ability of the article, potentially resulting in premature leakage. Additionally, it is desirable to attach components in such a way that the overall integrity (i.e. no loose or missing pieces) of the articles is preserved.

Various techniques are available for attaching components to the chassis and for attaching components to one another. Traditionally, adhesive application was the predominant technique for attaching components. More recently, adhesive application has been replaced by molecular bonding with ultrasonic bonding representing one specific method of molecular bonding. In very simplified terms, ultrasonic bonding involves the vibration of a “horn” device at ultrasonic frequencies which, in combination with an “anvil”, causes localized heating and bonding of substrate material(s) positioned between the horn and the anvil. This localized heating and bonding forms a bond element when a raised projection is provided on either the horn or the anvil. The raised projection forms an individual bond element in approximately the shape of the raised projection, and leaves the surrounding material relatively unchanged. Representative examples of rotary ultrasonic horns which have been used to bond at least one component are described in U.S. Pat. No. 5,096,532 to Neuwirth et al. on Mar. 17, 1992 and U.S. Pat. No. 5,110,403 issued to Ehlert on May 5, 1992. A representative example of a rotary ultrasonic bonding apparatus is described in U.S. Pat. No. 6,537,401 issued to Couillard et al. on Mar. 25, 2003. A problem with current rotary ultrasonic bond patterns is “chatter” or “bounce” which results when a smooth horn is pressed against a patterned anvil. This “bounce” may cause wear of the anvil, horn or support structure of the rotary ultrasonic device. This wear can potentially cause wear and increased variability in bonding effectiveness.

The individual bond elements may be in the form of straight or curved lines, geometric shapes such as circles, squares, rectangles, diamonds, and similar shapes or irregular shapes. A bond pattern is formed when the bond element is one of a group of one or more bond elements spaced in relative proximity to each other. The bond pattern may be formed by individual bond elements arranged in a linear or nonlinear configuration. Such patterns may be located on the bonding horn or the anvil roll. However, the pattern is traditionally located on the anvil roll for improved manufacturing efficiency. Various ultrasonic bond patterns may be achieved through arrangement of appropriate raised projections on an anvil.

Conventional ultrasonic bond patterns have positioned adjacent bond elements in uniform straight lines and crossing rows for manufacturing efficiency. The design of these conventional bond patterns has evolved as a replacement for adhesive bonding. As the number and type of components increases and as new component materials become available, presently available bond patterns may not be optimal.

Adhesive bonding usually causes little or no damage to the materials being bonded together, and therefore, from a bond strength perspective, more adhesive per length or area is generally better. Due to the energy associated with forming ultrasonic bonds, ultrasonic bonding may weaken or damage materials. Therefore, in contrast, more ultrasonic bonding per length or area may cause degradation of the materials being bonded together if the ultrasonic bonds are too dense or too great in number. As a result, presently available bond patterns may not provide sufficient attachment strength.

Desirably, materials that are bonded together overlap minimally. Minimal overlap reduces raw material cost, as well as improves the aesthetics of the finished product. Minimal overlap is also beneficial when bonding stretchable components together because conventional bonding techniques (adhesive and ultrasonic) may tie up or prevent the material within the bond area from stretching as much as the unbonded material.

Adhesive bonding, unlike ultrasonic bonding, has the additional problem of overspray. Overspray occurs when misapplied adhesive is not contained between two components. This misapplied adhesive may then cause processing and product problems. The misapplied adhesive may cause the product, or other materials, to adhere to parts of the processing machine potentially resulting in a jam. The misplaced adhesive may also be exposed on the product after manufacturing which may cause the product to stick to itself, products to stick to one another, or product to stick to the user. Even though ultrasonic bonding has been perceived to overcome some of the challenges associated with adhesive attachment, opportunities to provide improved ultrasonic bonding patterns still exist.

Accordingly, there remains a need for ultrasonic bond patterns which bond components to the chassis of absorbent articles with a greater resistance to failure. There also remains a need for ultrasonic bond patterns that bond components to the chassis of absorbent articles with lower variability. Moreover, in some circumstances, there remains a need for ultrasonic bond patterns that bond stretchable materials in such a way so as to maximize the amount of stretch retained in the bond area.

### SUMMARY OF THE INVENTION

The present invention relates generally to the use of bond patterns for the bonding of components to the chassis of an absorbent article. As described herein in more detail, absorbent articles typically include three primary components: a bodyside liner, an absorbent core and an outer cover. The chassis of an article typically refers to the area defined by the bodyside liner and the outer cover. The bodyside liner and the outer cover may run generally contiguous with each other. Typically, the two-dimensional area of the absorbent core is somewhat less than the areas of the bodyside liner and outer cover and the absorbent core is located between the bodyside liner and the outer cover. Many absorbent articles have systems for the fastening of the article about a wearer. The fastening systems may include mechanical fasteners such as a hook material that engages a complementary loop material to join one waist region to the other. It is possible for the hook material to be located directly on the chassis materials of the article. More typically however, the hook material is located on a back or front ear portion that is attached to the chassis. The most common configuration is for the hook material to be located on a back ear portion for engagement with a complementary loop material located in the front waist region of the outer cover. Placement of the hook material on a back ear portion that extends laterally outward from the chassis facilitates joinder of the waist regions around a wearer of the article.

The present invention is directed in part to a disposable absorbent article that may include two lateral edges and two longitudinal edges. The absorbent article may also include a front waist region, a back waist region and a crotch region interconnecting the front waist region and the back waist region. In addition, the disposable absorbent article includes a bodyside liner, an outer cover, and an absorbent core. The bodyside liner is provided in superimposed relation to the outer cover and the absorbent core is disposed between the outer cover and the bodyside liner. The disposable absorbent article may also include an ear portion attached by a plurality of bond elements to one longitudinal edge. The ear portion may be attached in either the front waist region or the back waist region. The bond elements may form a bond pattern that defines a bond pattern area. The bond pattern area may have a Percent Stretch of the Bond Pattern Area from about 8% to about 30%. The bond elements may be formed using known techniques such as ultrasonic, adhesive, thermal, laser and pressure. The Percent Stretch of the Bond Pattern Area is determined by measuring the un-tensioned bond pattern width; measuring the bond pattern width when loaded to an equivalent of 1500 gram/three inches; subtracting the un-tensioned width from the loaded width; dividing by the un-tensioned width; and multiplying the quotient by 100. Alternatively, the bond pattern area may have a Percent Stretch of the Bond Pattern Area from about 10% to about 20%.

In another aspect of the present invention, the bond elements may form a bond pattern which defines a bond pattern area. The percentage of the bond pattern area that may be bonded may be referred to as the percent bonded area. The percent bonded area may be defined as the area of the bond elements divided by the bond pattern area. The bond pattern area may be defined by the product of the bond pattern width and the bond pattern length. Preferably, the bond pattern length is the length of the bonded portion that includes a whole number of repeating sequences of bond pattern elements. For example, if the bond pattern elements are arranged to form a repeating sequence of circle, circle, square, a bond pattern length may include a whole number multiple of this sequence. The bond pattern length is ideally chosen to be about 75 millimeters, but desirably corresponds with a multiple of the repeating of the bond elements. The percent bonded area may be between about 5% and about 25%. In another aspect of the present invention, the percent bond area may be between about 10% and about 20%. In another aspect, the un-tensioned bond pattern area width may be between about 10 mm and about 18 mm. Alternatively the un-tensioned bond pattern area width may be between about 12 mm and about 16 mm.

In another aspect of the invention the ear may also be attached to the longitudinal edge of the article with multiple individual adhesive ribbons. The individual adhesive ribbon width may be less than 7 mm. The bond elements may be formed using known techniques such as ultrasonic, adhesive, thermal, laser and pressure.

In yet another aspect of the present invention, the bond pattern area may have a Percent Stretch of the Bond Pattern Area from about 8% to about 30% and the bond pattern may have a bond pattern edge and a Bond Pattern Integrity value of from 0.30 to 0.60. The Bond Pattern Integrity value is defined as the quotient of the fracture line length that is formed by the edges of bond elements and the entire fracture line length. The fracture line is defined as a path of least resistance that travels along an edge of the bond pattern. Alternatively, the bond pattern may have a Bond Pattern Integrity value from about 0.35 to about 0.55, or from about 0.40 to about 0.55, or from about 0.45 to about 0.55.

The bond pattern may also be characterized by the bond element area which may impact the path of least resistance and therefore may impact the Bond Pattern Integrity Value. The bond elements may have a bond element area of greater than about 0.007585 in². Alternatively, the bond elements may have a bond element area of less than about 0.007585 in². Alternatively, the bond element may have a bond element area of less than about 0.00210 in². The bond pattern may also be characterized by the center-to center spacing between the bond elements. The bond pattern may have a center-to-center spacing along the bond pattern edge of greater than about 0.500 inches. Alternatively, the bond pattern can have a center-to-center spacing along the bond pattern edge of between about 0.500-0.250 inches or a center-to-center spacing along the bond pattern edge of less than about 0.250 inches. The bond element may be formed using known techniques such as adhesive, thermal, laser and pressure. The bond element may have a circular, diamond, or square shape. Additionally the bond element may have a regular or an irregular shape.

In another aspect, the present invention relates to a disposable-absorbent article that includes two lateral edges, two longitudinal edges, a front waist region, a back waist region and a crotch region interconnecting the front waist region and back waist region. In addition, the disposable absorbent article includes a bodyside liner, an outer cover, and an absorbent core. The bodyside liner is provided in superimposed relation to the outer cover and the absorbent core is disposed between the outer cover and the bodyside liner. The disposable absorbent article may also include an ear portion attached by a plurality of bond elements to a longitudinal edge. The bond elements may form a bond pattern where the bond pattern includes two or more pairs of longitudinal rows of bond elements. The longitudinal rows of bond elements may be offset in the longitudinal direction by about 30% to about 70% of a longitudinal length of an individual bond element. There may be three or more, four or more, or five or more pairs of longitudinal rows of bond elements. The longitudinal rows of bond elements may be offset in the longitudinal direction by about 40% to about 60% of a longitudinal length of an individual bond element. Alternatively, the longitudinal rows of bond elements may be offset in the longitudinal direction from by about 45% to about 55% of a longitudinal length of an individual bond element.

It is to be understood that both the foregoing general description and the following detailed description are exemplary and are intended to provide further explanation of the invention claimed. The accompanying drawings, that are incorporated in and constitute part of this specification, are included to illustrate and provide a further understanding of the articles of the invention. Together with the description, the drawings serve to explain various aspects of the invention.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively shows a perspective view of an example of a disposable absorbent article (an infant diaper) of the present invention;

FIG. 2 representatively shows a plan view of the disposable absorbent article of FIG. 1 in an unfastened, stretched and laid flat condition with the surface of the article which contacts the wearer's skin facing the viewer and with portions of the article partially cut away to show the underlying features;

FIG. 3 representatively shows a perspective view of another example of a disposable absorbent article (a diaper pant) of the present invention;

FIG. 4 representatively shows a plan view of the disposable absorbent article of FIG. 3 in an unfastened, stretched and laid flat condition with the surface of the article which contacts the wearer's skin facing the viewer and with portions of the article partially cut away to show the underlying features;

FIG. 5 representatively shows a plan view of an example of an attachment area between a back ear portion and the longitudinal side edge of the chassis of a disposable absorbent article including a variegated bond pattern and a uniform bond pattern;

FIG. 6 representatively shows a plan view of a representative example of a bond pattern, such as may be used to bond ear portions of the disposable absorbent articles of the invention;

FIGS. 7A, 7B, and 7C representatively show examples of bond patterns with approximately equal-sized round bond elements with superimposed fracture lines along possible paths of least resistance;

FIGS. 8A, 8B, 8C, 8D, 8E, 8F, and 8G representatively show the patterned anvils (at a magnification of 5.3×) used to prepare samples of the present invention;

FIGS. 9A and 9B representatively show examples of bond patterns that may be described as being offset from each other.

### DETAILED DESCRIPTION OF THE INVENTION

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is directed to solving problems related to absorbent articles including the durability and integrity with which components, such as ear portions, are attached to the chassis of the articles. Additionally, the present invention is directed to improving the durability and integrity with which components, such as ear portions, are attached to each other. The present invention is also directed to maintaining the stretch of the bonded areas used to attach components, such as ear portions to the chassis of the articles. This detailed description of the present invention will include a description of a representative absorbent article including the various components of such articles. The description of the representative absorbent article will also include a description of the features encompassed by the present invention.

Representative Absorbent Article

The absorbent articles of the present invention will be described in terms of a disposable diaper article and in terms of a diaper pant that is adapted to be worn by infants about the lower torso. It is understood that the features of the present invention are equally adaptable for other types of absorbent articles such as adult incontinence pads, adult incontinence garments, training pants, disposable swim pants and feminine hygiene pads.

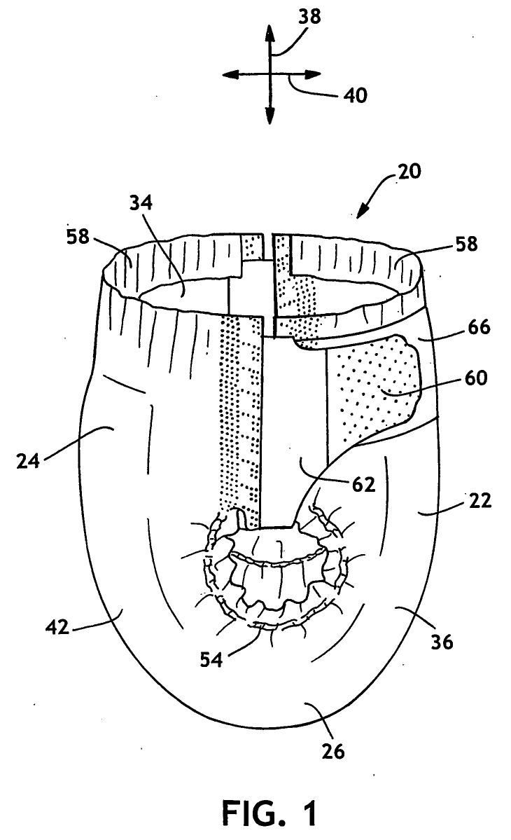

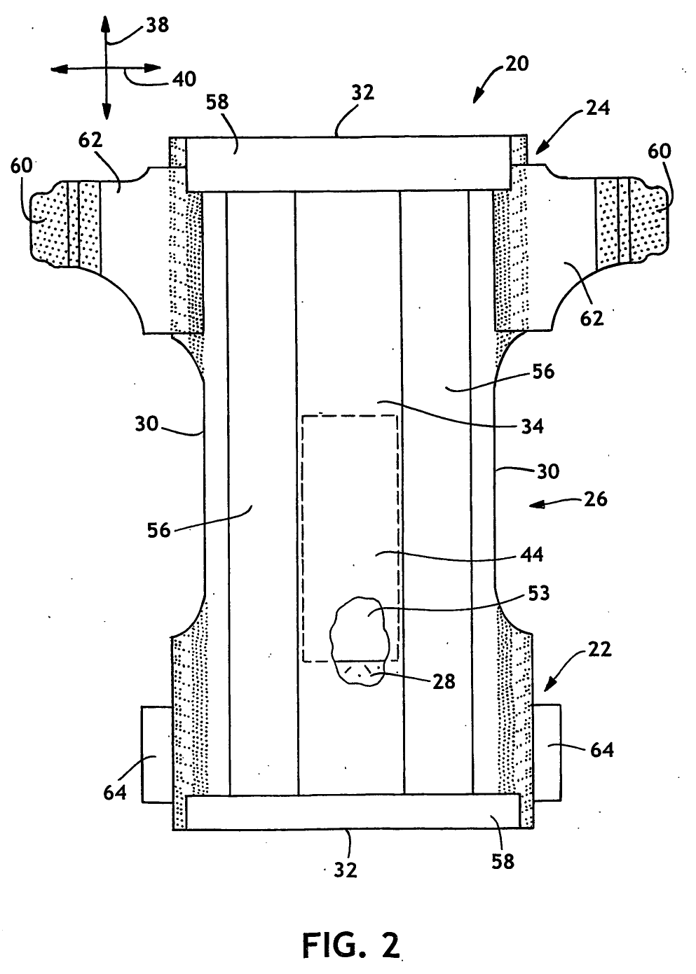

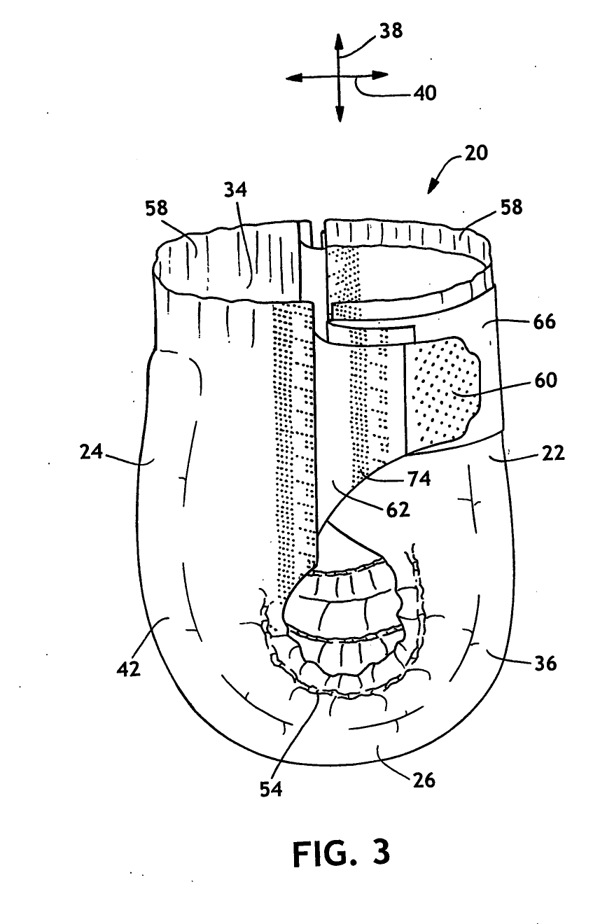

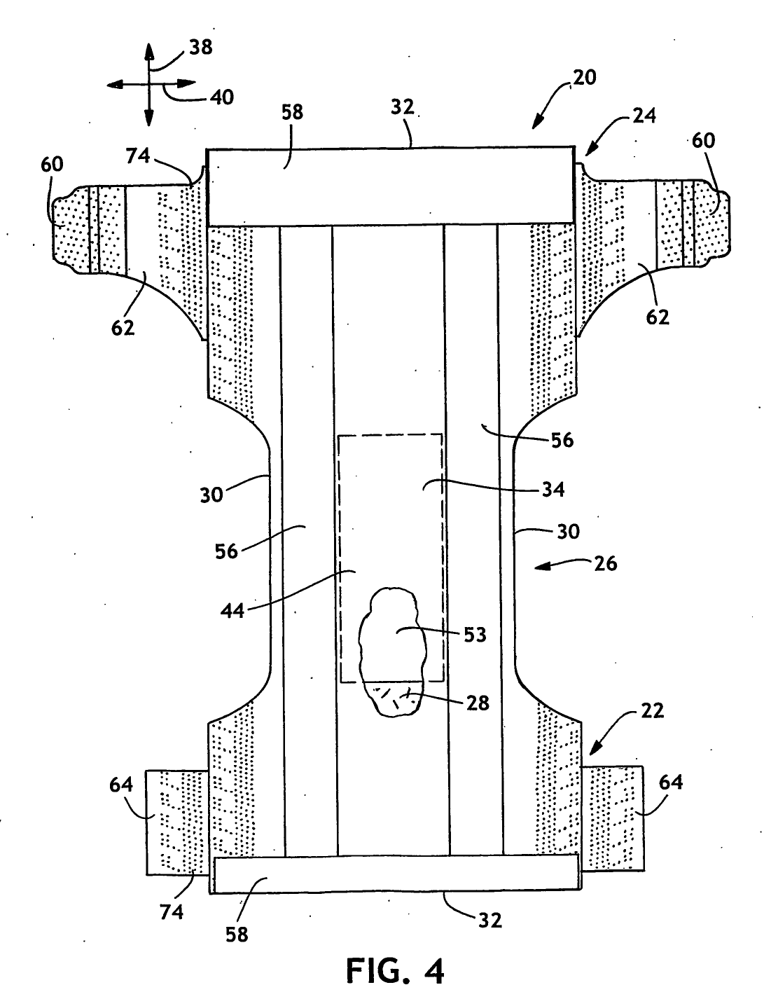

FIG. 1 representatively illustrates an example of a refastenable disposable diaper, as generally indicated at 20, of the present invention. FIG. 2 representatively illustrates the refastenable diaper of FIG. 1 in an unfastened, stretched and laid flat configuration with the surface of the diaper adapted to contact the wearer's skin facing the viewer and with portions of the diaper partially cut away to show the underlying features. FIG. 3 representatively illustrates another example of an absorbent article of the invention, a diaper pant generally indicated at 20. FIG. 4 representatively illustrates the prefastened diaper pant of FIG. 3 in an unfastened, stretched and laid flat configuration with the surface of the diaper pant adapted to contact the wearer's skin facing the viewer and with portions of the diaper pant partially cut away to show the underlying features. As illustrated in FIG. 2 and FIG. 4, the diaper/diaper pant 20 defines a front waist region 22, a back waist region 24, a crotch region 26 that extends between and connects the front and back waist regions 22 and 24, a longitudinal direction 38 and a lateral direction 40. The front waist region 22 includes the portion of the diaper/diaper pant 20 that, when worn, is positioned on the front of the wearer while the back waist region 24 includes the portion of the diaper/diaper pant 20 that, when worn, is positioned on the back of the wearer. The crotch region 26 of the diaper/diaper pant 20 includes the portion of the diaper/diaper pant 20 that, when worn, is positioned between the legs of the wearer and covers the lower torso of the wearer.

The diaper/diaper pant 20 defines a pair of laterally opposed side edges 30, a pair of longitudinally opposed waist edges 32, an interior surface 34 that is configured to contact the wearer, and an exterior surface 36 opposite the interior surface 34 that is configured to contact the wearer's clothing in use. The illustrated diaper/diaper pant 20 also includes a substantially liquid impermeable outer cover 42 and a liquid permeable bodyside liner 44 that can be connected to the outer cover 42 in a superposed relation. An absorbent core 28 is located between the outer cover 42 and the bodyside liner 44. The laterally opposed side edges 30 of the diaper/diaper pant 20 are generally defined by the side edges of the outer cover 42 that further define leg openings that may be curvilinear. The waist edges 32 of the diaper/diaper pant 20 are generally defined by the waist edges of the outer cover 42 and define a waist opening that is configured to encircle the waist of the wearer when worm. The absorbent core 28 is configured to contain and/or absorb body exudates discharged from the wearer. The diaper/diaper pant 20 may further include leg elastics 54, containment flaps 56 and waist elastics 58 as are known to those skilled in the art. It should be recognized that individual components of the diaper/diaper pant 20 may be optional depending upon the intended use of the diaper/diaper pant 20.

The diaper/diaper pant 20 may further include refastenable mechanical fasteners 60. The mechanical fasteners 60 releasably engage the opposed side edges 30 of the diaper/diaper pant 20 in the opposite waist regions. The mechanical fasteners 60 can include a variety of materials and surfaces known for mechanical engagement such as buttons, pins, snaps, adhesive tape fasteners, cohesives, mushroom-and-loop fasteners and hook and loop fasteners. Further, the disposable diaper/diaper pant 20 may include an attachment panel 66 located on the front or back waist region 22 and 24, opposite the fasteners 60 to which the fasteners 60 can be releasably engaged during use of the diaper/diaper pant 20.

The diaper/diaper pant 20 may be of various suitable shapes. For example, in the unfastened configurations as illustrated in FIG. 2 and FIG. 4, the diaper/diaper pant 20 may have an overall rectangular shape, T-shape or an approximately hourglass shape. In the shown embodiments, the diaper/diaper pant 20 has a generally I-shape in an unfastened configuration.

The various components of the diaper/diaper pant 20 are integrally assembled together employing various types of suitable attachment means, such as adhesive, sonic and thermal bonds or combinations thereof. In the shown embodiments, for example, the outer cover 42 and bodyside liner 44 are assembled to each other and to the absorbent core 28 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent core 28 may be connected to the outer cover 42 using conventional fasteners such as buttons, hook and loop type fasteners, adhesive tape fasteners, and the like. The other components of the diaper/diaper pant 20 may be suitably connected together using similar means. Similarly, other diaper components, such as the elastic members 54 and 58 and the mechanical fasteners 60, may be assembled into the diaper/diaper pant 20 article by employing the above-identified attachment mechanisms. Desirably, the majority of the diaper components are assembled together using ultrasonic bonding techniques for reduced manufacturing cost.

The outer cover 42 of the diaper/diaper pant 20, as representatively illustrated in FIGS. 1 and 3, may suitably be composed of a material which is either liquid permeable or liquid impermeable. It is generally preferred that the outer cover 42 be formed from a material that is substantially impermeable to liquids. A typical outer cover 42 can be manufactured from a thin plastic film or other flexible liquid-impermeable material. For example, the outer cover 42 may be formed from a polyethylene film having a thickness of from about 0.013 millimeter (0.5 mil) to about 0.051 millimeter (2.0 mils). The materials of the outer cover 42 can be thermally or adhesively laminated together. Suitable laminate adhesives, which can be applied continuously or intermittently as beads, a spray, parallel swirls, or the like, can be obtained from Bostik-Findley, Inc., of Wauwatosa, Wis., U.S.A., or from National Starch and Chemical Company, Bridgewater, N.J., U.S.A. If it is desired to present the outer cover 42 with a more clothlike feeling, the outer cover 42 may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may be thermally laminated thereto a spunbond web of polypropylene fibers. The polypropylene fibers may have a fiber diameter of about 15 to 20 microns, which nonwoven web has a basis weight of about 17 grams per square meter (0.5 ounce per square yard). The outer cover 42 may include bicomponent fibers such as polyethylene/polypropylene bicomponent fibers. Methods of forming such clothlike outer covers are known to those skilled in the art. The outer cover 42 may also be an extensible outer cover such as the outer covers described in U.S. Pat. No. 6,552,245 issued on Apr. 22, 2003 to Roessler et al. The outer cover 42 may also be a biaxially stretchable outer cover such as the outer covers described in U.S. patent application Ser. No. 09/698,517 filed on Oct. 27, 2000 by Vukos et al.

The outer cover 42 may be formed of a woven or nonwoven fibrous web layer which has been totally or partially constructed or treated to impart a desired level of liquid impermeability to selected regions that are adjacent or proximate the absorbent core 28. Still further, the outer cover 42 may optionally be composed of a micro-porous “breathable” material which permits vapors to escape from the absorbent core 28 while still preventing liquid exudates from passing through the outer cover 42. For example, the outer cover 42 may include a vapor permeable non-woven facing layer laminated to a micro-porous film. Suitable “breathable” outer cover materials are described in U.S. Pat. No. 5,695,868 issued Dec. 9, 1997 to McCormack et al. and U.S. Pat. No. 5,843,056 issued Dec. 1, 1998 to Good et al., the descriptions of which are hereby incorporated by reference. Still further, the outer cover 42 may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. The outer cover 42 can also be embossed or otherwise provided with a matte finish to provide a more aesthetically pleasing appearance.

In order to reduce the perception that the outer cover 42 feels damp or clammy, the diapers/diaper pants 20 of the invention may include a spacer or ventilation layer (not shown in Figures) between the garment-facing surface of the absorbent core 28 and the outer cover 42. The ventilation layer may include one or more nonwoven materials, for example a spunbond-meltblown-spunbond nonwoven material.

The representative absorbent articles of the invention include a bodyside liner 44 in superimposed relation to the outer cover 42. The bodyside liner 44, as representatively illustrated in FIG. 2 and FIG. 4, suitably presents a bodyfacing surface that is compliant, soft feeling, and nonirritating to the wearer's skin. Further, the bodyside liner 44 may be less hydrophilic than the absorbent core 28, to present a relatively dry surface to the wearer, and may be sufficiently porous to be liquid permeable, permitting liquid to readily penetrate through its thickness. A suitable bodyside liner 44 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 44 is suitably employed to help isolate the wearer's skin from liquids held in the absorbent 28. The bodyside liner 44 can also be made from extensible materials as are described in U.S. Pat. No. 6,552,245 issued on Apr. 22, 2003 to Roessler et al. The bodyside liner 44 can also be made from biaxially stretchable materials as are described in U.S. patent application Ser. No. 09/698,517 filed on Oct. 27, 2000 by Vukos et al.

Various woven and nonwoven fabrics can be used for the bodyside liner 44. For example, the bodyside liner may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In a particular embodiment of the present invention, the bodyside liner 44 is made from a nonwoven, spunbond, polypropylene fabric composed of fibers having a fiber diameter of about 21 to 23 microns formed into a web having a basis weight of about 20 grams per square meter and a density of about 0.13 grams per cubic centimeter. The fabric may be surface treated with about 0.3 weight percent of a surfactant, such as a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 44 or may be selectively applied to particular sections of the bodyside liner 44, such as the medial section along the longitudinal centerline of the diaper, to provide greater wettability of such sections. The bodyside liner 44 may further include a lotion or treatment applied thereto that is configured to be transferred to the wearer's skin. Suitable compositions for application to the bodyside liner 44 are described in U.S. Pat. No. 6,149,934 that issued to Krzysik et al. on Nov. 21, 2000.

The representative absorbent articles of the invention can include an absorbent core 28 disposed between the outer cover 42 and the bodyside liner 44. The absorbent core 28 of the diaper/diaper pant 20, as representatively illustrated in FIG. 1 and FIG. 3, may suitably include a matrix of hydrophilic fibers, such as a web of cellulosic fluff, mixed with particles of a high-absorbency material commonly known as superabsorbent material. In a particular aspect, the absorbent core 28 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent core 28 may include a laminate of fibrous webs and superabsorbent material or other suitable matrix for maintaining a superabsorbent material in a localized area.

The absorbent core 28 may have any of a number of shapes. For example, the absorbent core 28 may be rectangular, I-shaped, or T-shaped. It is generally preferred that the absorbent core 28 is narrower in the intermediate section than in the front or rear waist sections of the diaper 20. The absorbent core 28 may be provided by a single layer or, in the alternative, may be provided by multiple layers, all of which need not extend the entire length and width of the absorbent core 28. In a particular aspect of the invention, the absorbent core 28 can be generally T-shaped with the laterally extending cross-bar of the “T” generally corresponding to the front waist region 22 of the absorbent article for improved performance, especially for male infants.

The size and the absorbent capacity of absorbent core 28 should be compatible with the size of the intended wearer and the liquid loading imparted by the intended use of the absorbent article. Further, the size and the absorbent capacity of the absorbent core 28 can be varied to accommodate wearers ranging from infants through adults. In addition, it has been found that with the present invention, the densities and/or basis weights of the absorbent core 28 can be varied.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent core 28 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. In general, the high absorbency material is present in the absorbent core 28 in an amount of from about 5 to about 90 percent by weight, desirably in an amount of at least about 30 percent by weight, and even more desirably in an amount of at least about 50 percent by weight based on a total weight of the absorbent core 28. For example, in a particular aspect, the absorbent core 28 may include a laminate which includes at least about 50 percent by weight and desirably at least about 70 percent by weight of high-absorbency material overwrapped by a fibrous web or other suitable material for maintaining the high-absorbency material in a localized area.

An example of high-absorbency material suitable for use in the present invention is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

Optionally, a substantially hydrophilic tissue or nonwoven wrapsheet (not illustrated) may be employed to help maintain the integrity of the structure of the absorbent core 28. The wrapsheet is typically placed about the absorbent core 28 over at least the two major facing surfaces thereof. The wrapsheet may be composed of an absorbent cellulosic material, such as creped wadding or a high wet-strength tissue. In one aspect of the invention, the wrapsheet may be configured to provide a wicking layer that helps to rapidly distribute liquid over the mass of absorbent fibers constituting the absorbent core 28.

Due to the thinness of absorbent core 28 and the high absorbency material within the absorbent core 28, the liquid uptake rates of the absorbent core 28, by itself, may be too low, or may not be adequately sustained over multiple insults of liquid into the absorbent core 28. To improve the overall liquid uptake and air exchange, the diaper/diaper pant 20 of the different aspects of the present invention may further include a porous, liquid-permeable layer of surge management material 53, as representatively illustrated in FIG. 2 and FIG. 4. The surge management layer 53 is typically less hydrophilic than the absorbent core 28, and has an operable level of density and basis weight to quickly collect and temporarily hold liquid surges, to transport the liquid from its initial entrance point and to substantially completely release the liquid to other parts of the absorbent core 28. This configuration can help prevent the liquid from pooling and collecting on the portion of the diaper/diaper pant 20 positioned against the wearer's skin, thereby reducing the feeling of wetness by the wearer. The structure of the surge management layer 53 also generally enhances the air exchange within the diaper/diaper pant 20.

Various woven and nonwoven fabrics can be used to construct the surge management layer 53. For example, the surge management layer 53 may be a layer composed of a meltblown or spunbond web of synthetic fibers, such as polyolefin fibers. The surge management layer 53 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 53 may be composed of a substantially hydrophobic material, and the hydrophobic material may optionally be treated with a surfactant or otherwise processed to impart a desired level of wettability and hydrophilicity. In a particular aspect, the surge management layer 53 includes a hydrophobic, nonwoven material having a basis weight of from about 30 to about 120 grams per square meter.

The absorbent articles of the invention can include additional components. For example, as representatively illustrated in FIGS. 14, the disposable diaper/diaper pant 20 may include a pair of containment flaps 56 that are configured to provide a barrier to the lateral flow of body exudates. The containment flaps 56 may be located along the laterally opposed side edges 30 of the diaper/diaper pant adjacent the side edges of the absorbent core 28. Each containment flap 56 typically defines an unattached edge that is configured to maintain an upright, perpendicular configuration in at least the crotch region 26 of the diaper/diaper pant 20 to form a seal against the wearer's body. The containment flaps 56 may extend longitudinally along the entire length of the absorbent core 28 or may only extend partially along the length of the absorbent core 28. When the containment flaps 56 are shorter in length than the absorbent core 28, the containment flaps 56 can be selectively positioned anywhere along the side edges 30 of diaper/diaper pant 20 in the crotch region 26. In a particular aspect of the invention, the containment flaps 56 extend along the entire length of the absorbent core 28 to better contain the body exudates. Such containment flaps 56 are generally well known to those skilled in the art.

The diaper/diaper pant 20 of the different configurations of the present invention may further include elastics at the waist edges 32 and side edges 30 of the diaper/diaper pant 20 to further prevent leakage of body exudates and support the absorbent core 28. For example, as representatively illustrated in FIGS. 1-4, the diaper/diaper pant 20 of the present invention may include a pair of leg elastic members 54 that are connected to the laterally opposed side edges 30 of the diaper/diaper pant 20 in the crotch region 26. The diaper/diaper pant 20 may also include a pair of waist elastic members 58 that is connected to the longitudinally opposed waist edges 32 of the diaper/diaper pant 20. The leg elastics 54 and waist elastics 58 are generally adapted to fit about the legs and waist of a wearer in use to maintain a positive, contacting relationship with the wearer to effectively reduce or eliminate the leakage of body exudates from the diaper/diaper pant 20.

Materials suitable for use as the leg elastics 54 and waist elastics 58 are well known to those skilled in the art. Exemplary of such materials are sheets or strands or ribbons of a polymeric, elastomeric material that may be adhered to the outer cover 42 in a stretched position, or that may be attached to the outer cover 42 while the outer cover is pleated, such that elastic constrictive forces are imparted to the outer cover 42. The leg elastics 54 may also include such materials as polyurethane, synthetic and natural rubber. The waist elastics 58 may be formed by elastic strands attached to the outer cover 42 or they may be formed by attaching separate pieces of stretchable materials to the waist regions of the article. For example, the waist elastics 58 may include a piece of stretch-bonded laminate material attached to the interior surface 34 of the article to form a waistband. Elasticity may be added or incorporated into the waist opening of absorbent articles utilizing a variety of known approaches.

The absorbent articles of the invention may include one or more components that extend laterally outward from the longitudinal sides of the article. Typically, the longitudinal sides are defined by the materials forming the chassis of the diaper/diaper pant 20. The chassis may be defined by the outer cover 42 and bodyside liner 44 materials. Components that extend laterally outward may include front ear portions 64 and back ear portions 62. The front ear portions 64 and the back ear portions 62 may be formed of one or more materials and may include laminates of materials. The front ear portions 64 and the back ear portions 62 improve the fit of the absorbent article. More specifically, the front ear portions 64 may provide additional coverage around the waist of the wearer and they may assist caregivers with positioning the front waist region 22 on the wearer of the article. The front ear portions 64 may also include mechanical fastening materials such that the front ear portions 64 contribute to the overall fastening system of the article. The back ear portions 62 may also provide coverage around the waist of the wearer. More specifically, the back ear portions 62 may provide the bridging material between the back waist region 24 of the article and the front waist region 22 such that the back ear portions 62 form part of the article's waist opening and an upper edge of the article's leg openings. Additionally, the back ear portions 62 may include fastening materials that facilitate joining of the back waist region 24 with the front waist region 22. For example, the back ear portions 62 may include fasteners 60 selected for engagement with an attachment panel 66 in the front waist region 22 of the article.

Presently available infant diapers typically include back ear portions 62 that include a stretchable material. When the back ear portions 62 include a stretchable material, the back ear portions 62 may increase the range with which the fasteners 60 may be engaged into the attachment panel 66 or directly into the outer cover 42. Further, when the back ear portions 62 include a stretchable material, the article may be worn by a greater range of users as a result of the increased fit range. An exemplary material from which the back ear portions 62 may be constructed is a necked bonded laminate material having two nonwoven (e.g. spunbond) facings with an elastomeric film (e.g. KRATON film) laminated in between. Other suitable stretchable materials are known in the art. Depending on the design of the article, it may also be desirable for the front ear portions 64 to include a stretchable material.

When the product form of the absorbent article is a training pant or a swim pant, the back ear portions 62 and the front ear portions 64 are understood to include the side panels that are attached to the longitudinal sides 30 of the article and also are attached to each other to form side seams of the article. Typically, the side panels of training pants and swim pants are made from stretchable materials. The side panels' ability to stretch allows these products to be pulled on the wearer like underpants.

The diaper pant 20 form of the present invention (representatively illustrated in FIG. 3 and FIG. 4) may be described as a hybrid between an infant diaper that is typically removed and applied while the child is lying down and a training pant that is put on like underpants. The product form may be referred to as a diaper pant because the diaper pant may be applied and removed as either a diaper or a pant. A diaper pant may have a back ear portion 62 and a front ear portion 64 where the back ear portion 62 and front ear portion 64 are attached to each other by a passive side bond 74. The passive side bond 74 may be selected to be readily tearable by caregivers during the process of “converting” the diaper pant from a pant to a diaper. Desirably, the passive side bond 74 is easily opened/broken without tearing of the materials used to form the back ear portion 62 and the front ear portion 64.

With each of the product forms, the back ear portions 62 and the front ear portions 64 may be attached to the longitudinal side edges 30 of the article by bonding techniques, such as ultrasonic bonding. Use of ultrasonic bonding techniques tends to form discrete bond points. The bond patterns of the invention, as will be discussed herein, may be formed using known techniques such as adhesive, thermal, laser and pressure that are capable of forming the patterns. Ultrasonic bonding will be referred to for purposes of example. With articles of the invention, the back ear portions 62 and the front ear portions 64 may be attached to one or more of the chassis materials using one or more bond patterns. The articles of the invention may include an attachment area 76 where the attachment area 76 includes an overlapping area of the material forming the outer cover 42 and the material forming either a back ear portion 62 or a front ear portion 64. The attachment area 76 may further include a variegated bond pattern 70 and a uniform bond pattern 72 where each bond pattern provides attachment between the outer cover 42 material and the ear portion material. The attachment area 76 may also include an overlapping area of the material forming the bodyside liner 44 and the material forming either a back ear portion 62 or a front ear portion 64. Additionally, the attachment area 76 may include an overlapping area of the material forming the outer cover 42, the material forming the bodyside liner 44 and the material forming either a back ear portion 62 or a front ear portion 64.

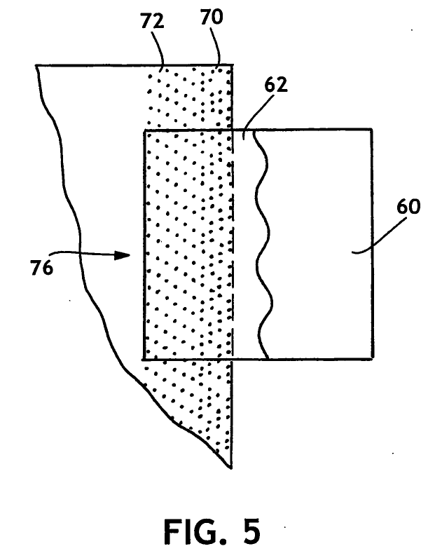

The variegated bond pattern 70 may be formed by a plurality of bond points that are variable in location with respect to each other. Put differently, the bond points of the variegated bond pattern 70 may not all be located equidistantly from each other. While the bond points of the variegated bond pattern 70 may spaced irregularly with respect to each other, the variegated bond pattern 70 may have a repeating pattern appearance. The uniform bond pattern 72 may be formed by a plurality of bond points that are regular in location with respect to each other. Put differently, the bond points of the uniform bond pattern 72 are located generally equidistantly from each other. The variegated bond pattern 70 and the uniform bond pattern 72 may be immediately adjacent to each other as illustrated in FIG. 5 or the patterns may have some open space in between them. The bond patterns may be formed by ultrasonic bonds or by other bonding techniques capable of providing individual bond points.

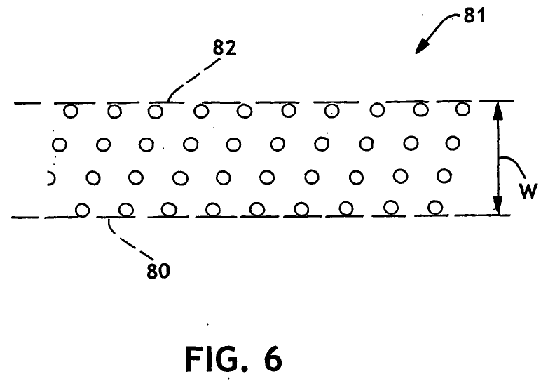

FIG. 6 illustrates a representative example of a bond pattern 81 that may be selected for attaching a back ear portion 62 to the chassis of a diaper 20. In addition to ear portions 62/64 being attached to the chassis through the use of variegated bond pattern 70 and uniform bond patterns 72, bond patterns may be selected to provide increased bond strength and tear resistance.

A bond pattern may be understood to include a plurality of bond elements located in general proximity to each other (such as the array of bond element s shown in FIG. 6). The bond pattern may be defined by its dimensions. For example, a bond pattern 81 width “W” may be defined by the distance in the lateral direction 40 between the first edge 80 of the bond pattern, and the second edge 82 of the bond pattern. A bond pattern width can be measured at any point along the length of the bond pattern. The bond pattern edge may be curvy which may result in the bond pattern width varying along the length of the bond pattern. When a bond pattern has curvy edge, the bond pattern width may be defined as the average bond pattern width. The bond pattern edge may be curvy when three or more consecutive bond elements are located away from a straight bond pattern edge.

A bond element area is defined for non-hollow bond elements 85 as the geometric area of the bond element 85. For hollow bond elements, such as a bond element in the shape of a “donut”, the bond element area is defined as the area enclosed by the outer circumference of the bond element. For a “donut” shaped bond element this area would include the area of the “donut hole.” All bond elements 85 shown in all figures are non-hollow.

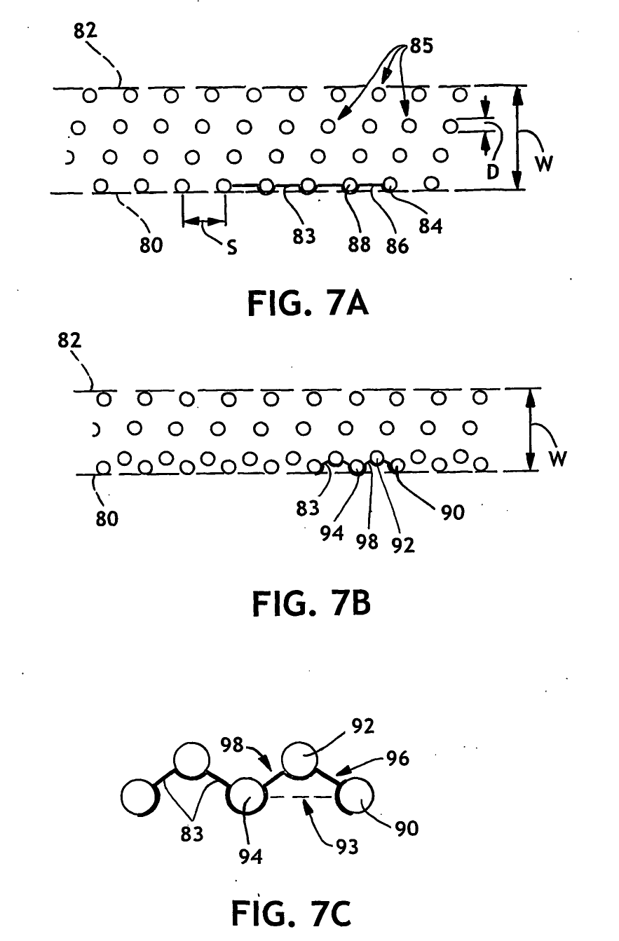

If a bond pattern 81 securing an ear portion 62 to an absorbent article (FIG. 7A) is susceptible to failure, the failure is likely to occur along a path of least resistance. The path of least resistance may correspond to portions of bond elements' circumferences. The path of least resistance may also contain portions corresponding to spaces between bond elements 86. This path of least resistance may propagate along either edge 80, 82 of the bond pattern 81.

FIG. 7A representatively illustrates an example of a bond pattern 81 with approximately equal sized round, non-hollow bond elements 85 superimposed with a possible fracture line 83 following a path of least resistance along a first edge 80 of the bond pattern. The fracture line 83 may extend the entire length of the first edge 80 of the bond pattern, however only the fracture line 83 between five bond elements 85 is shown in FIG. 7A. The fracture line 83 begins at a location where the circumference of a first bond element 84 contacts the first edge 80 of the bond pattern. The fracture line 83 may follow the circumference of the first bond element 84 away from the first edge 80 of the bond pattern until the fracture line 83 is perpendicular to the shortest line 86 connecting the first bond element 84 and a second bond element 88. The fracture line 83 may then follow this shortest line 86 until the fracture line 83 intersects with the circumference of the second bond element 88. The fracture line then follows the circumference of the second bond element 88 towards the intersection of the circumference of the second bond element 88 with the first edge 80 of the bond pattern. The fracture line 83 may continue in this manner along the first edge 80 of the bond pattern. Hence, the fracture line 83 may contain portions formed by the circumferences of bond elements as well as portions formed by the straight lines between bond elements 84, 88. The relationship of the portions formed by the circumferences of bond elements 84, 88 and of the portions formed by the straight lines 86 between bond elements 84, 88 is described in detail below.

FIG. 7B representatively illustrates a second example of a bond pattern with approximately equal sized round, non-hollow bond elements superimposed with a possible fracture line 83 following a path of least resistance along a first edge 80 of the bond pattern. As with the example shown in FIG. 7A, the fracture line 83 may extend the entire length of the first edge 80 of the bond pattern, however, only a portion the fracture line 83 between five bond elements is shown in FIG. 7B. In addition, a blow up of the five bond elements from FIG. 7B is shown in FIG. 7C. In this example, as above, the fracture line 83 may start where the edge of a first bond element 90 contacts the first edge 80 of the bond pattern. The fracture line 83 may then follow the circumference of the bond element 90 away from the contact with the first edge 80 of the bond pattern until the fracture line 83 contacts the shortest segment 96 connecting the first bond element 90 and a second bond element 92 that is not located on the first edge 80 of the bond pattern. The fracture line 83 may then follow this first shortest segment 96 until the fracture line 83 intersects with the circumference of the second bond element 92. The fracture line 83 may then follow the circumference of the second bond element 92 starting in the direction of the first edge 80 of the bond pattern and continuing until the fracture line 83 contacts the shortest segment 98 connecting the second bond element 92 and a third bond element 94 located on the first edge 80 of the bond pattern. The fracture line 83 may then follow this shortest segment 98 until the fracture line 83 intersects with the circumference of the third bond element 94. The fracture line 83 may then follow the circumference of the third bond element 94 towards the intersection of the third bond element 94 with the first edge 80 of the bond pattern. The fracture line 83 may then repeat this pattern.

This fracture line 83 may travel from the first bond element 90 located on the first edge 80 of the bond pattern to the second bond element 92 not located on the first edge 80 of the bond pattern if this is the path of least resistance. The fracture line 83 in FIG. 7B will be the path of least resistance if the sum of the length of the shortest segment 96 between the first bond element 90 and the second bond element 92 and the length of the shortest segment 98 between the second bond element 92 and the third bond element 94 is less then the length of the shortest segment 93 between the first bond element 90 and the third bond element 94. If the sum of the length of segment 96 and the length of segment 98 is equal to or greater than the length of segment 93, the fracture line 83 will likely not travel to a bond element not located on the first edge 80 of the bond pattern; instead, the fracture line will travel in the manner shown in FIG. 7A.

Desirably, the bond patterns of the invention balance the contributions to the integrity of the bonded composite by design of the bond pattern. More specifically the invention balances contributions from bond elements and contributions from the spaces between bond elements. With respect to the fracture line 83, it is desirable to balance the portion of the fracture line 83 that may travel along the circumference of bond elements with the portion of the fracture line 83 that may travel between bond elements (traveling through unbonded material). More specifically, when the length of the fracture line that travels along the circumference of bond elements is equal to the length of the fracture line that travels between bond elements, a maximum bond strength may be attained. The maximum bond strength occurs when a Bond Pattern Integrity Value is equal to 0.500.

The Bond Pattern Integrity Value is defined as the quotient of the fracture line length that is formed by the edges of bond elements and the entire fracture line length. For example, for circular bond elements having diameter “D” (see FIG. 7A) and a fracture line 83 that travels between bond elements located on the edge of the bond width, with center-to-center spacing “S” (see FIG. 7A), the calculation for the Bond Pattern Integrity Value is: [((Π*D)/2)/(((Π*D)/2)+S−D)].

In a second example, the bond elements 85 may be square-shaped and have a side length “L”. For square shaped bond elements 85, the fracture line may travel between bond elements 85 located on the edge of the bond pattern, with a side parallel to the edge of the bond pattern, and with center-to-center spacing “S”, the calculation for the Bond Pattern Integrity Value is: [L/S].

In a third example, the bond elements 85 may be square-shaped and have a side length “L”. For square shaped bond elements 85, the fracture line may travel between bond elements 85 located on the edge of the bond pattern, with a side 45 degrees to the edge of the bond pattern (diamond orientation), and with center-to-center spacing “S”, the calculation for the Bond Pattern Integrity Value is: [(2*L)/((2*L)+S−(L* √2))].

These exemplary calculations are representative of how the Bond Pattern Integrity Value may be calculated for bond patterns of the invention. For more complex geometries, measurement using optical methods known in the art may be used to determine the Bond Pattern Integrity Value.

The Bond Pattern Integrity Values and the strength of the bonds of bond patterns 81 representing bond patterns of the invention were determined. In addition, the Bond Pattern Integrity Values of presently available commercial bond patterns were measured. For example PAMPERS NOCTURNA diapers (available from the Procter & Gamble Co.) purchased in Brazil, in June 2003, and PAMPERS Premium diapers (available from the Procter & Gamble Co.) purchased in the United States, in June 2003 were tested. These commercially available diapers included back ear portions that are bonded to the longitudinal edge of the chassis of diapers.

The Bond Pattern Integrity Value was determined by first measuring required lengths, radii, and angles of the bond elements of the bond pattern from bonded materials. A SWIFT microscope model # 3208 was used to make the measurements of the bond patterns. The measurements were made at 4× magnification. The ocular used was a SWIFT POINTMASTER ocular EW 10×D/20.50 14.5 MM, which is a scaled ocular that was calibrated to a graduated slide with a 2 mm measurement divided into units of 0.01 mm. The slide was manufacture by American Optical of Southbridge, Mass. This calibration indicated that 67 gradations equaled 2 mm. Pictures may be taken for irregular shaped bonds and image analysis may be used to calculate the dimensions needed when the picture contains a reference such as some measurement device in the picture. A person skilled in the art will recognize many equivalent methods possible of attaining similar results.

From these measurements, the Bond Pattern Integrity Value may be calculated, either with one of the above formulas, an appropriate formula for the geometry of the bond elements and bond pattern, or a physical measurement of the portions of the fracture line. The Bond Pattern Integrity Values calculated for the PAMPERS diapers products are provided in Table 3. below. In addition to measuring aspects of the bond patterns used on PAMPERS diapers, the bond element diameter “D” for circular bond elements or side length “L” for square bond elements, and center-to center spacing “S” for bond patterns of the invention were determined and are provided in Table 1. below.

All examples of bond patterns of the invention were prepared by placing 4.3 osy NBL between spunbond reversibly neck-stretched liner material and a poly spunbond outer cover material, such as that used on HUGGIES Supreme diapers and ultrasonically bonding with a plunge bonder having a flat horn and a patterned anvil. The neck-bonded laminate material is representatively of a suitable material that may be used to form an ear portion 62.

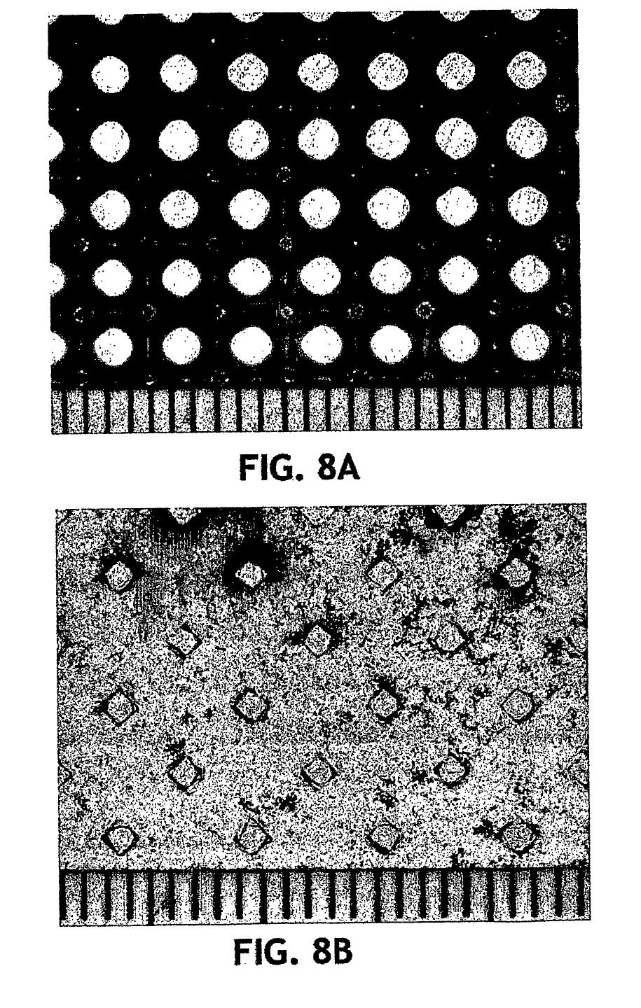

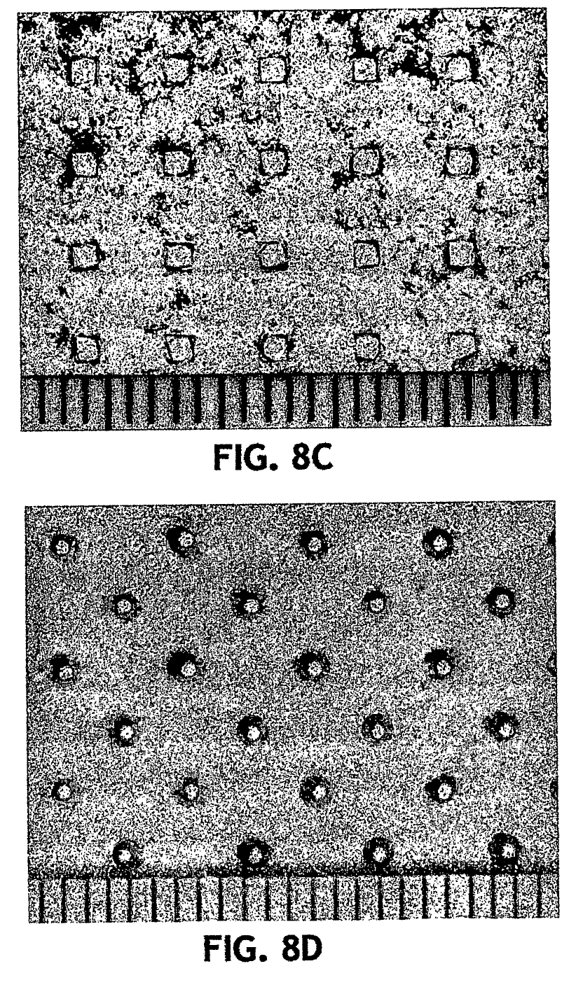

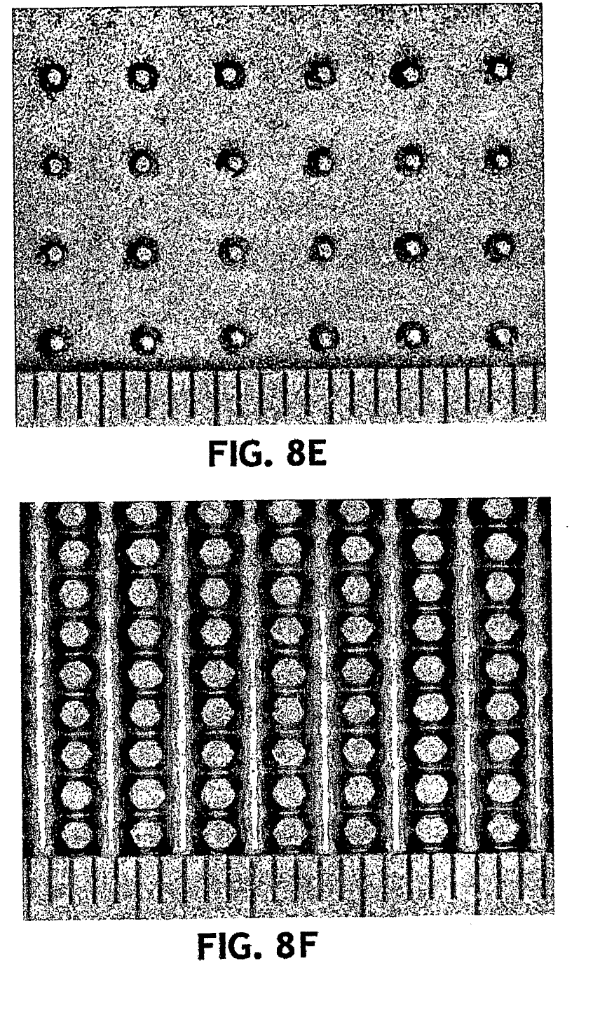

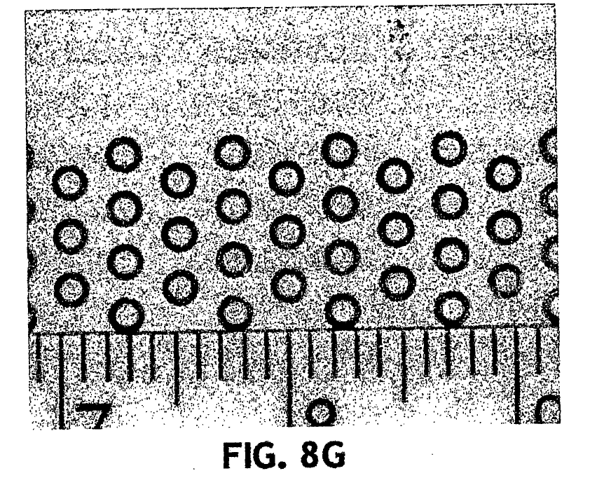

The patterned anvils used to make the samples of the invention were magnified 5.3 times and photographed. These photographs are shown in FIGS. 8A to 8G. The patterned anvil of Code A is shown in FIG. 8A. The patterned anvil of Code B is shown in FIG. 8B. The patterned anvil of Code C is shown in FIG. 8C. The patterned anvil of Code D is shown in FIG. 8D. The patterned anvil of Code E is shown in FIG. 8E. The patterned anvil of Code F is shown in FIG. 8F. The patterned anvil of Code G is shown in FIG. 8G.

TABLE 1

| | | | Center-to-Center |
|---|---|---|---|
| | | Diameter (Round) | Spacing Along Edge |
| | Bond Element | Length of Side | of the Bond |
| Code | Geometry | (Square) (mm) | Pattern (mm) |
| A | Round | 1.641 | 3.125 |
| B | Square | 1.104 | 5.912 |
| | (Diamond | | |
| | Orientation) | | |
| C | Square | 1.104 | 4.167 |
| D | Round | 0.746 | 5.594 |
| E | Round | 0.746 | 3.972 |
| F | Round | 1.194 | 3.158 |
| G | Round | 0.985 | 2.350 |

In addition to measuring the parameters relevant to calculate the Bond Pattern Integrity Value, the bond patterns of the invention may also be characterized by the strength of the bonds. In order to test the strength of the bond between the materials bonded with the bond patterns of the invention, Codes A-G were tested according to ASTM test procedure D-5733-99 modified in two ways, from which a stress-strain curve was calculated. The first modification of ASTM D-5733-99 is the elimination of the initiating cut. The second modification of ASTM D-5733-99 is the placement of the jaws to be parallel with the edges of the bond pattern one inch from the edge of the bond pattern. The data recorded in Table 2. below indicates the Bond Energy of the bond patterns. The Bond Energy is the amount of energy required to completely rupture the bonds, which is represented by the area under the stress-strain curve. The strength of the bonds is a function of many factors. Some of these factors are the chemical composition of the material being bonded, material weight, horn amplitude and Bond Pattern Integrity Value. For this reason, comparison of the strength of the bond for samples made with non-uniform materials may not be useful. For Codes A-G, the same material and material basis weights were used. Additionally, the same bonder was used. The Bond Pattern Integrity Values for PAMPERS Noturna, PAMPERS Premium diapers, and HUGGIES Supreme diapers (available from the Kimberly-Clark Corp) were determined and are recorded in Table 3. below.

TABLE 2

| | Bond Pattern Integrity Value | Bond Energy |
|---|---|---|
| Code | (Calculated Based on Values in Table 1.) | (grams*cm) |
| A | 0.635 | 16392 |
| B | 0.337 | 17779 |
| C | 0.265 | 13497 |
| D | 0.195 | 5959 |
| E | 0.267 | 4655 |
| F | 0.488 | 20483 |
| G | 0.531 | 12978 |

TABLE 3

| Code | Bond Pattern Integrity Value |
|---|---|
| PAMPERS Noturna diapers | 0.679 |
| PAMPERS Premium diapers | 0.830 |
| HUGGIES Supreme diapers | 0.608 |

The Bond Pattern Integrity Value approaches a minimum of zero when the circumference of the bond elements 85 get small and the center-to-center spacing gets large. Consequently, there are very few bond elements 85 along the fracture line 83 to bond the material together. Additionally, the Bond Pattern Integrity Value approaches a maximum of one when the circumference of the bond elements 83 gets large and the center-to-center spacing gets small. When this occurs, the fracture line 83 exists entirely on the circumference of bond elements 85. Added strength is achieved when the fracture line 83 makes the transition from a bond element circumference to the material in between bond elements 85. Up to a point the more transitions per length of bonded material will increase the strength of the bond. Additionally, an optimum Bond Pattern Integrity Value exists for a given bond set-up of a given material and a given bonding technique. For a given bond pattern set-up, as the Bond Pattern Integrity Value increases from zero, there is more bonding holding the materials together and the strength of the bond pattern increases. For the same bond pattern set-up, as the Bond Pattern Integrity Value approaches one, there is less and less material between bonds to provide strength and the strength of the bond decreases. The Bond Pattern Integrity Value for bond pattern of the invention may range from about 0 to 1, more specifically from about 0.2 to 0.8, and more specifically from about 0.3 to 0.7.

In addition to the Bond Pattern Integrity Value, another aspect relating to the orientation and the spacing of the bond elements 85 of bond patterns is the percent bonded area.

The percent bonded area may be defined as the area of bond elements divided by the bond pattern area. The bond pattern area may be defined by the product of the bond pattern width and the bond pattern length. The bond pattern width is described previously herein. The bond pattern length may be the entire length of the bond pattern that is on the absorbent article. Preferably, the bond pattern length is the length of the bonded portion that includes a whole number of repeating sequences of bond pattern elements. For example, if the bond pattern elements are arranged to form a repeating sequence of circle, circle, square, a bond pattern length would include a whole number multiple of this sequence. To determine the area of bond elements of a bond pattern with only one type of bond element, the dimensions of the bond element are determined using the method described above, and the area of the bond element is multiplied by the number of bond elements in the bond pattern area. The bond pattern length is ideally chosen to be about 75 millimeters, but desirably corresponds with a multiple of the repeating of the bond elements.

Desirably, absorbent articles are formed from extensible and stretchable materials so as to improve the fit of the articles. A stretchable material (such as may be used to form an ear portion 62/64 is a material that is capable of extending upon application of a tensile force and capable of retracting either partially or close to completely to its original dimension(s) upon removal of the force depending on the desired use within the absorbent article. An extensible material is a material that is capable of extending upon application of a tensile force, but the extension is permanent. Stretchable and extensible materials may be more expensive than non-stretchable and non-extensible materials. Desirably, the inclusion of stretch or extensibility to absorbent articles using these materials may be optimized by minimizing the reduction in stretch or extensibility that may be caused by bonding. A measure of the amount of stretch or extensibility remaining in the components after bonding is the percent stretch of the bond pattern area.

The percent stretch of a bond pattern area may be determined by first marking the edges of the bond pattern of a bonded area of materials from an absorbent article. The bonded area of materials is then cut into a three inch by approximately six inch rectangle. The rectangle is cut in such a way that the bond length on the rectangle is three inches and the bond pattern area is approximately centered in the six inch dimension. A first three inch clamp is applied to one edge of the rectangle, such that the clamp is attached parallel to the edge of the bond pattern, approximately one inch away from the first edge of the bond pattern. The first three inch clamp is then attached to a hanger which allows the rectangle to hang from the first three inch clamp. The distance between the marks is measured indicating the un-stretched bond pattern width. A second three inch clamp that has been weighted to a total of 1500 grams is then attached parallel to the second edge of the bond pattern opposite the first three inch clamp, approximately one inch away from the second edge of the bond pattern. The clamps are now aligned away from the bond pattern allowing room to measure between the marks. After the weight has been allowed to hang for 10 seconds, the distance between the marks is measured indicating the stretched bond pattern width. By subtracting the un-stretched bond pattern width from the stretched bond pattern width, then dividing by the un-stretched bond pattern width and multiplying the quotient by 100, the percent stretch of the bond pattern area may be determined. If the bonded area of material has a bond less than three inches in length, the width and weight of the second clams may be adjusted to deliver an equivalent loading 1500 gram/three inches.

The percent bonded area, bond energy, and percent stretch of the bond pattern area of Codes A-G were determined via the methods described above and are provided in Table 4. below.

TABLE 4

| | | | Percent Stretch of |
|---|---|---|---|
| | Percent Bonded Area | Bond Energy | the Bond Pattern Area |
| Code | (%) | (grams*cm) | (%) |
| A | 15.9 | 16392 | 10.5 |
| B | 6 | 17779 | 25.7 |
| C | 6.2 | 13497 | 20.0 |
| D | 4.8 | 5959 | 17.5 |
| E | 4.6 | 4655 | 23.2 |
| F | 20.2 | 20483 | 5.0 |
| G | 15.7 | 12978 | 8.3 |

For a given bond pattern set-up, as the percent bond area increases, there is more area to contribute to the integrity of the bond pattern and the bond energy generally increases. For the same bond pattern set-up, as the percent bond area increases, there is less free material to allow the bond area to stretch and the percent stretch of the bond pattern area generally decreases. The percent bond area may range from 0 to 100, more specifically form 1 to 50, more specifically from 2 to 25, and more specifically from 3 to about 20.

In addition to Bond Pattern Integrity Value and percent bond area, a third important aspect relating to the orientation and spacing of the bond elements of bond patterns is the percent offset in the longitudinal direction 38.

Both Bond Pattern Integrity Value and percent bond area, when changed, have an immediate effect on attributes of the bond pattern 81. However with percent offset in the longitudinal direction, a change does not have an immediate effect. However, percent offset in the longitudinal direction does have an effect over time. As previously described, a problem with current rotary bond patterns is “chatter” or “bounce” that may result when a smooth horn is pressed against a patterned anvil. This “bounce” may cause wear of the anvil, horn or support structure of the rotary bond device. This wear may cause wear in the rotary bonding device and increased variability in bonding effectiveness. Percent offset in the longitudinal direction addresses this problem.

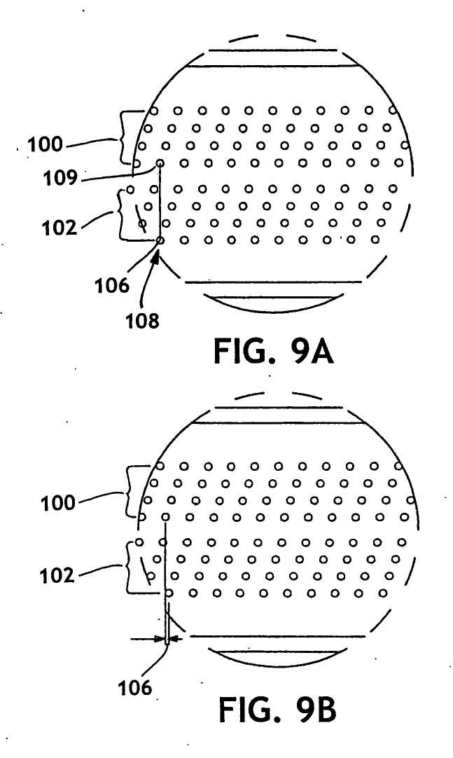

The percent offset in the longitudinal direction is determined by first measuring the length of a first bond element 108 (FIG. 9A) in the longitudinal direction, as described previously herein. Second a bond element 109 of the same size and shape is located in a different longitudinal row of the bond pattern. The offset is the length that the second bond element 109 overlaps the first bond element 108 in the longitudinal direction divided by the length of the bond element in the longitudinal direction. This offset is multiplied by 100 to give a percentage. FIG. 9A shows a bond pattern with 100% overlap 106 between a first set of bond elements 100 and a second set of bond elements 102. In FIG. 9B, the first set of bond elements 100 has been offset from the second set of bond elements 102 by 50%, indicated by overlap 106. Offset may be achieved by this method of offsetting a first set from a second set. In FIG. 9B four rows of bond elements are offset from four other rows of bond elements, forming four pairs. Alternatively, the offset rows need not be in separate sets. A pair of offset rows may be located adjacent to each other or alternatively they may be separated by one or more rows. The rows of this pair are then no longer considered in determining how many pairs of rows are offset. This offset reduces the amount of wear that the horn and anvil are exposed to, and therefore provides for less variability in bonding conditions and bond strength over time.

Delivering the bond patterns of the present invention in absorbent articles provide several benefits including improved fit and improved performance. The higher stretch of the bond patterns improve the utilization of the stretchable materials used in absorbent articles, allowing lesser quantities of materials to be used. The higher strengths and reduced variability of the strengths of the bond patterns of the present invention provide improved durability to the absorbent articles.

It will be appreciated that details of the bond patterns of the invention, given for purposes of illustration, are not to be construed as limiting the scope of this invention. Although only a few exemplary aspects of this invention have been described in detail above, those skilled in the art will readily appreciate that many modifications are possible in the exemplary aspects without materially departing from the novel teachings and advantages of this invention. Accordingly, all such modifications are intended to be included within the scope of this invention, which is defined in the following claims and all equivalents thereto. Further, it is recognized that many aspects may be conceived that do not achieve all of the advantages of some aspects, particularly of the preferred aspects, yet the absence of a particular advantage should not be construed to necessarily mean that such an aspect is outside the scope of the present invention.

1. A disposable absorbent article including two lateral edges, two longitudinal edges, a front waist region, a back waist region and a crotch region interconnecting the front waist region and back waist region, the disposable absorbent article further comprising:
a bodyside liner;
an outer cover;
an absorbent core located between the bodyside liner and the outer cover; and
an ear portion attached by a plurality of bond elements to one longitudinal edge, wherein the bond elements form a bond pattern which defines a bond pattern area and wherein the bond pattern area has a Percent Stretch of the Bond Pattern Area of from 8% to 30%.

2. The disposable absorbent article of claim 1, wherein the bond pattern has a bond pattern edge and a Bond Pattern Integrity value of from 0.30 to 0.60.

3. The disposable absorbent article of claim 2, wherein the bond elements are ultrasonic bond elements.

4. The disposable absorbent article of claim 2, wherein the bond elements have a circular shape.

5. The disposable absorbent article of claim 2, wherein the bond pattern has a Bond Pattern Integrity value of from 0.35 to 0.55.

6. The disposable absorbent article of claim 2, wherein the bond pattern has a Bond Pattern Integrity value of from 0.40 to 0.55.

7. The disposable absorbent article of claim 2, wherein the bond pattern has a Bond Pattern Integrity value of from 0.45 to 0.55.

8. The disposable absorbent article of claim 2, where the bond elements have a bond element area of greater than 0.007585 in².

9. The disposable absorbent article of claim 2, where the bond pattern has a Bond element area of less than 0.007585 in².

10. The disposable absorbent article of claim 2, where the bond elements have a bond element area less than 0.00210 in².

11. The disposable absorbent article of claim 2, where the bond pattern has a center-to-center spacing along the bond pattern edge of greater than 0.500 in.

12. The disposable absorbent article of claim 2, where the bond pattern has a center-to-center spacing along the bond pattern edge of between 0.500-0.250 in.

13. The disposable absorbent article of claim 2, where the bond pattern has a center-to-center spacing along the bond pattern edge of less than 0.250 in.

14. The disposable absorbent article of claim 1, wherein the bond elements are ultrasonic bond elements.

15. The disposable absorbent article of claim 1, where the bond pattern area has a Percent Stretch of the Bond Pattern Area from 10% to 20%.

16. The disposable absorbent article of claim 1, wherein the bond pattern area has an untensioned width between 10-18 mm.

17. The disposable absorbent article of claim 1, where the bond pattern area has an untensioned width between 12-16 mm.

18. The disposable absorbent article of claim 1, where the percent bonded area is between 5% and 25%.

19. The disposable absorbent article of claim 1, where the percent bonded area is between 10% and 20%.

20. The disposable absorbent article of claim 1, where the ear portion is also attached to the longitudinal edge with multiple individual adhesive ribbons, wherein a width of the individual adhesive ribbons is less than 7 mm.

21. A disposable absorbent article including two lateral edges, two longitudinal edges, a front waist region, a back waist region and a crotch region interconnecting the front waist region and back waist region, the disposable absorbent article further comprising:
a bodyside liner;
an outer cover;
an absorbent core located between the bodyside liner and the outer cover; and
an ear portion attached by a plurality of ultrasonic bonds to one longitudinal edge of the disposable absorbent article, wherein the ultrasonic bonds form a bond pattern and wherein the bond pattern includes two or more pairs of longitudinal rows of bond elements, wherein the longitudinal rows of bond elements are offset in the longitudinal direction from 30% to 70% of a longitudinal length of an individual bond element.

22. The disposable absorbent article of claim 21, wherein there are three or more pairs of longitudinal rows of bond elements.

23. The disposable absorbent article of claim 21, wherein there are four or more pairs of longitudinal rows of bond elements.

24. The disposable absorbent article of claim 21, wherein there are five or more pairs of longitudinal rows of bond elements.

25. The disposable absorbent article of claim 21, wherein the longitudinal rows of bond elements are offset in the longitudinal direction from 40% to 60% of a longitudinal length of an individual bond element.

26. The disposable absorbent article of claim 21, wherein the longitudinal rows of bond elements are offset in the longitudinal direction from 45% to 55% of a longitudinal length of an individual bond element.
